(19) United States
(12) Patent Application Publication
(10) Pub. No.: US 2018/0213818 A1
(43) Pub. Date: Aug. 2, 2018

(54) PRODUCTION OF SOLUBLE PROTEIN PRODUCTS FROM HEMP ("H701")

(71) Applicant: Burcon NutraScience (MB) Corp., Winnipeg (CA)

(72) Inventors: Martin Schweizer, Winnipeg (CA); Brandy Gosnell, Winnipeg (CA)

(73) Assignee: Burcon NutraScience (MB) Corp., Winnipeg (CA)

(21) Appl. No.: 15/938,210

(22) Filed: Mar. 28, 2018

Related U.S. Application Data
(63) Continuation of application No. 13/956,619, filed on Aug. 1, 2013, now abandoned.
(60) Provisional application No. 61/678,722, filed on Aug. 2, 2012.

(51) Int. Cl. A23J 1/14; C07K 14/415; A23J 1/00; A23L 2/66

(52) U.S. Cl. CPC A23J 1/14; C07K 14/415; A23V 2002/00; A23L 2/66; A23J 1/007

(57) ABSTRACT

A hemp protein product, which may be an isolate, produces solutions at low pH values and is useful for the fortification of soft drinks and sports drinks without precipitation of protein. The hemp protein product is obtained by extracting a hemp protein source material with an aqueous calcium salt solution to form an aqueous hemp protein solution, separating the aqueous hemp protein solution from residual hemp protein source, adjusting the pH of the aqueous hemp protein solution to a pH of about 1.5 to about 4.4 to produce an acidified hemp protein solution, which may be dried, following optional concentration and diafiltration, to provide the hemp protein product.

### REFERENCE TO RELATED APPLICATION

This application is Continuation of U.S. application Ser. No. 13/956,619 filed Aug. 1, 2013 and claims priority under 35 USC 119(e) from U.S. Application No. 61/678,722 filed Aug. 2, 2012.

### FIELD OF INVENTION

The present invention is directed to the production of protein product from hemp and to novel hemp protein products.

### BACKGROUND TO THE INVENTION

In U.S. patent application Ser. No. 12/603,087 filed Oct. 21, 2009 (US Patent Publication No. 2010-0098818), Ser. No. 12/923,897 filed Oct. 13, 2010 (US Patent Publication No. 2011-0038993) and Ser. No. 12/998,422 filed Jun. 1, 2011 (US Patent Publication No. 2011-0236556), assigned to the assignee hereof and the disclosures of which are incorporated herein by reference, there is described the production of soy protein products having a protein content of at least about 60 wt % (N×6.25) d.b., preferably at least about 90 wt %, which produce transparent, heat stable solutions at low pH values and which may be used for protein fortification of soft drinks, as well as other aqueous systems, without precipitation of protein.

The soy protein product is produced by extracting a soy protein source with an aqueous calcium chloride solution to cause solubilization of soy protein from the protein source and to form an aqueous soy protein solution, separating the aqueous soy protein solution from residual soy protein source, optionally diluting the soy protein solution, adjusting the pH of the aqueous soy protein solution to a pH of about 1.5 to about 4.4, preferably about 2 to about 4, to produce an acidified clear soy protein solution, optionally concentrating the aqueous clear protein solution while maintaining the ionic strength substantially constant by using a selective membrane technique, optionally diafiltering the concentrated soy protein solution, and optionally drying the concentrated and optionally diafiltered soy protein solution.

### SUMMARY OF THE INVENTION

It has been found that this procedure and modifications thereof, may be used to form acid soluble protein products from hemp having a protein content of at least 60 wt % (N×6.25) d.b. The acid soluble hemp protein products may be used for protein fortification of, in particular, soft drinks and sports drinks, more particularly powdered soft drinks and sports drinks, which are dissolved in water by the end user, as well as other aqueous systems, without precipitation of protein.

The novel hemp protein product is completely soluble in aqueous solution at acid pH values less than about 4.4. Given the complete solubility of the product, no stabilizers or other additives are necessary to maintain the protein in solution or suspension. The product is low in phytic acid, generally less than about 1.5 wt %, preferably less than about 0.5 wt %. No enzymes are required in the production of the hemp protein product. The hemp protein product has been described as having a bland flavor. The hemp protein product is preferably an isolate having a protein content of at least about 90 wt %, preferably at least about 100 wt % (N×6.25).

In accordance with one aspect of the present invention, there is provided a method of producing a hemp protein product having a hemp protein content of at least about 60 wt % (N×6.25) on a dry weight basis, which comprises:
(a) extracting a hemp protein source with an aqueous calcium salt solution, preferably aqueous calcium chloride solution, to cause solubilization of hemp protein from the protein source and to form an aqueous hemp protein solution,
(b) separating the aqueous hemp protein solution from residual hemp protein source,
(c) optionally diluting the aqueous hemp protein solution,
(d) adjusting the pH of the aqueous hemp protein solution to a pH of about 1.5 to about 4.4, preferably about 2 to about 4, to produce an acidified hemp protein solution,
(e) optionally clarifying the acidified hemp protein solution if it is not already clear,
(f) alternatively from steps (b) to (e), optionally diluting and then adjusting the pH of the combined aqueous hemp protein solution and residual hemp protein source to a pH of about 1.5 to about 4.4, preferably about 2 to about 4, then separating the acidified, preferably clear, hemp protein solution from residual hemp protein source,
(g) optionally concentrating the aqueous hemp protein solution while maintaining the ionic strength substantially constant by using a selective membrane technique,
(h) optionally diafiltering the concentrated hemp protein solution, and
(i) optionally drying the concentrated and optionally diafiltered hemp protein solution.

The hemp protein product preferably is an isolate having a protein content of at least about 90 wt %, preferably at least about 100 wt %, (N×6.25) d.b.

The present invention further provides a novel hemp protein product having a protein content of at least 60 wt %, preferably at least about 90 wt %, more preferably at least about 100 wt % (N×6.25) d.b., and which is water soluble at acid pH values of less than about 4.4 and is useful for the protein fortification of aqueous systems, including soft drinks and sports drinks, particularly powdered versions of these drinks, without leading to protein precipitation. The hemp protein product is also low in phytic acid content, generally less than about 1.5% by weight, preferably less than about 0.5% by weight. The hemp protein in the product is not hydrolyzed.

Thus, in another aspect to the present invention, there is provided a hemp protein product having a protein content of at least about 60 wt %, preferably a hemp protein isolate having a protein content of at least about 90 wt % (N×6.25) d.b., more preferably at least about 100 wt % (N×6.25) d.b., which is substantially completely soluble in an aqueous medium at a pH of less than about 4.4, preferably about 1.5 to about 4.4.

The hemp protein product provided herein may be provided as an aqueous solution thereof, preferably having a high degree of clarity at acid pH values, generally from less than about 4.4, preferably about 1.5 to about 4.4.

The novel hemp protein product of the invention can be blended with powdered drinks for the formation of aqueous soft drinks or sports drinks by dissolving the same in water. Such blend may be a powdered beverage. The novel hemp protein product may also be utilized in applications having a near neutral pH of about 6 to about 8.

While the present invention refers mainly to the production of hemp protein isolate, it is contemplated that hemp protein products of lesser purity may be provided having similar properties to the hemp protein isolate. Such lesser purity products may have a protein concentration of at least about 60% by weight (N×6.25) d.b.

In another aspect of the present invention, there is provided an acidic aqueous solution of the hemp protein product provided herein. The acidic aqueous solution may be a beverage, which may be a clear beverage in which the hemp protein product is completely soluble and transparent or the acidic aqueous solution may be a non-transparent beverage in which the hemp protein product does or does not contribute to the cloud in the beverage. The acidic aqueous solutions have good flavour attributes and, in informal taste panel tests, exhibited a blander taste than an aqueous solution of a commercial hemp protein product.

The hemp protein product produced according to the process herein is suitable, not only for protein fortification of acid media, but may be used in a wide variety of conventional applications of protein products, including but not limited to protein fortification of processed foods and beverages, emulsification of oils, as a body former in baked goods and foaming agent in products which entrap gases. In addition, the hemp protein product may be formed into protein fibers, useful in meat analogs and may be used as an egg white substitute or extender in food products where egg white is used as a binder. The hemp protein product may also be used as a nutritional supplement. The hemp protein product also may be used in dairy analogue or alternative products or products which are dairy/hemp blends. Other uses of the hemp protein product are in pet foods, animal feed and in industrial and cosmetic applications and in personal care products.

### GENERAL DESCRIPTION OF INVENTION

The initial step of the process of providing the hemp protein product involves solubilizing hemp protein from a hemp protein source. The hemp protein source may be hemp seeds or any hemp product or by-product derived from the processing of hemp seeds, including but not limited to hemp meal, hemp protein products made by sifting hemp meal and dehulled hemp seeds. The hemp protein source may be used in the full fat form, partially defatted form or fully defatted form. Where the hemp protein source contains an appreciable amount of fat, an oil-removal step generally is required during the process. The hemp protein recovered from the hemp protein source may be the protein naturally occurring in hemp or the proteinaceous material may be a protein modified by genetic manipulation but possessing characteristic hydrophobic and polar properties of the natural protein.

Protein solubilization from the hemp protein source material is effected most conveniently using calcium chloride solution, although solutions of other calcium salts, may be used. In addition, other alkaline earth metal compounds may be used, such as magnesium salts. Further, extraction of the hemp protein from the hemp protein source may be effected using calcium salt solution in combination with another salt solution, such as sodium chloride. Additionally, extraction of the hemp protein from the hemp protein source may be effected using water or other salt solution, such as sodium chloride, with calcium salt subsequently being added to the aqueous hemp protein solution produced in the extraction step. Precipitate formed upon addition of the calcium salt is removed prior to subsequent processing.

As the concentration of the calcium salt solution increases, the degree of solubilization of protein from the hemp protein source initially increases until a maximum value is achieved. Any subsequent increase in salt concentration does not increase the total protein solubilized. The concentration of calcium salt solution which causes maximum protein solubilization varies depending on the salt concerned. It is usually preferred to utilize a concentration value less than about 1.0 M, and more preferably a value of about 0.10 to about 0.15 M.

In a batch process, the salt solubilization of the protein is effected at a temperature of from about 1° C. to about 100° C., preferably about 15° to about 65° C., more preferably about 20° C. to about 35° C., preferably accompanied by agitation to decrease the solubilization time, which is usually about 1 to about 60 minutes. It is preferred to effect the solubilization to extract substantially as much protein from the hemp protein source as is practicable, so as to provide an overall high product yield.

In a continuous process, the extraction of the hemp protein from the hemp protein source is carried out in any manner consistent with effecting a continuous extraction of hemp protein from the hemp protein source. In one embodiment, the hemp protein source is continuously mixed with the calcium salt solution and the mixture is conveyed through a pipe or conduit having a length and at a flow rate for a residence time sufficient to effect the desired extraction in accordance with the parameters described herein. In such a continuous procedure, the salt solubilization step is effected in a time of about 1 minute to about 60 minutes, preferably to effect solubilization to extract substantially as much protein from the hemp protein source as is practicable. The solubilization in the continuous procedure is effected at temperatures between about 1° C. and about 100° C., preferably about 15° to about 65° C., more preferably between about 20° C. and about 35° C.

The extraction is generally conducted at a pH of about 4.5 to about 11, preferably about 5 to about 7. The pH of the extraction system (hemp protein source and calcium salt solution) may be adjusted to any desired value within the range of about 4.5 to about 11 for use in the extraction step by the use of any convenient food grade acid, usually hydrochloric acid or phosphoric acid, or food grade alkali, usually sodium hydroxide, as required.

The concentration of hemp protein source in the calcium salt solution during the solubilization step may vary widely. Typical concentration values are about 5 to about 15% w/v.

The protein extraction step with the aqueous salt solution has the additional effect of solubilizing fats which may be present in the hemp protein source, which then results in the fats being present in the aqueous phase.

The protein solution resulting from the extraction step generally has a protein concentration of about 5 to about 50 g/L, preferably about 10 to about 50 g/L.

The aqueous calcium salt solution may contain an antioxidant. The antioxidant may be any convenient antioxidant, such as sodium sulfite or ascorbic acid. The quantity of antioxidant employed may vary from about 0.01 to about 1 wt % of the solution, preferably about 0.05 wt %. The antioxidant serves to inhibit oxidation of any phenolics in the protein solution.

The aqueous phase resulting from the extraction step then may be separated from the residual hemp protein source, in any convenient manner, such as by employing a decanter centrifuge or any suitable sieve, followed by disc centrifugation and/or filtration, to remove residual hemp protein source material. The separation step may be conducted at any temperature within the range of about 1° to about 100° C., preferably about 15° to about 65° C., more preferably about 20° to about 35° C. Alternatively, the optional dilution and acidification steps described below may be applied to the mixture of aqueous hemp protein solution and residual hemp protein source, with subsequent removal of the residual hemp protein source material by the separation step described above. The separated residual hemp protein source may be dried for disposal. Alternatively, the separated residual hemp protein source may be processed to recover some residual protein. The separated residual hemp protein source may be re-extracted with fresh calcium salt solution and the protein solution yielded upon clarification combined with the initial protein solution for further processing as described below. Alternatively, the separated residual hemp protein source may be processed by a conventional isoelectric precipitation procedure or any other convenient procedure to recover residual protein.

The aqueous hemp protein solution may be treated with an anti-foamer, such as any suitable food-grade, non-silicone based anti-foamer, to reduce the volume of foam formed upon further processing. The quantity of anti-foamer employed is generally greater than about 0.0003% w/v. Alternatively, the anti-foamer in the quantity described may be added in the extraction steps.

Where the hemp protein source contains significant quantities of fat, as described in U.S. Pat. Nos. 5,844,086 and 6,005,076, assigned to the assignee hereof and the disclosures of which are incorporated herein by reference, then the defatting steps described therein may be effected on the separated aqueous protein solution. Alternatively, defatting of the separated aqueous protein solution may be achieved by any other convenient procedure.

The aqueous hemp protein solution may be treated with an adsorbent, such as powdered activated carbon or granulated activated carbon, to remove colour and/or odour compounds. Such adsorbent treatment may be carried out under any convenient conditions, generally at the ambient temperature of the separated aqueous protein solution. For powdered activated carbon, an amount of about 0.025% to about 5% w/v, preferably about 0.05% to about 2% w/v, is employed. The adsorbing agent may be removed from the hemp protein solution by any convenient means, such as by filtration.

The resulting aqueous hemp protein solution may be diluted generally with about 0.1 to about 10 volumes, preferably about 0.5 to about 2 volumes, of aqueous diluent in order to decrease the conductivity of the aqueous hemp protein solution to a value of generally below about 105 mS, preferably about 4 to about 21 mS. Such dilution is usually effected using water, although dilute salt solution, such as sodium chloride or calcium chloride, having a conductivity of up to about 3 mS, may be used.

The diluent with which the hemp protein solution is mixed generally has the same temperature as the hemp protein solution, but the diluent may have a temperature of about 1° to about 100° C., preferably about 15° to about 65° C., more preferably about 20° to about 35° C.

The optionally diluted hemp protein solution then is adjusted in pH to a value of about 1.5 to about 4.4, preferably about 2 to about 4, by the addition of any suitable food grade acid, such as hydrochloric acid or phosphoric acid, to result in an acidified aqueous hemp protein solution preferably a clear acidified aqueous hemp protein solution. The acidified aqueous hemp protein solution has a conductivity of generally below about 110 mS for a diluted hemp protein solution, or generally below about 115 mS for an undiluted hemp protein solution, in both cases preferably about 4 to about 26 mS.

As mentioned above, as an alternative to the earlier separation of the residual hemp protein source, the aqueous hemp protein solution and the residual hemp protein source material, may be optionally diluted and acidified together and then the acidified aqueous hemp protein solution is clarified and separated from the residual hemp protein source material by any convenient technique as discussed above. The acidified aqueous hemp protein solution may optionally be defatted, optionally treated with an adsorbent and optionally treated with defoamer as described above.

If the optionally diluted and acidified hemp protein solution is not transparent it may be clarified by any convenient procedure such as filtration or centrifugation.

If of adequate purity, the resulting acidified aqueous hemp protein solution may be directly dried to produce a hemp protein product. In order to provide a hemp protein product having a decreased impurities content and a reduced salt content, such as a hemp protein isolate, the acidified aqueous hemp protein solution may be processed as described below prior to drying.

The acidified aqueous hemp protein solution may be concentrated to increase the protein concentration thereof while maintaining the ionic strength thereof substantially constant. Such concentration generally is effected to provide a concentrated hemp protein solution having a protein concentration of about 50 to about 300 g/L, preferably about 100 to about 200 g/L.

The concentration step may be effected in any convenient manner consistent with batch or continuous operation, such as by employing any convenient selective membrane technique, such as ultrafiltration or diafiltration, using membranes, such as hollow-fibre membranes or spiral-wound membranes, with a suitable molecular weight cut-off, such as about 1,000 to about 1,000,000 Daltons, preferably about 1,000 to about 100,000 Daltons, having regard to differing membrane materials and configurations, and, for continuous operation, dimensioned to permit the desired degree of concentration as the aqueous protein solution passes through the membranes.

As is well known, ultrafiltration and similar selective membrane techniques permit low molecular weight species to pass therethrough while preventing higher molecular weight species from so doing. The low molecular weight species include not only the ionic species of the salt but also low molecular weight materials extracted from the source material, such as carbohydrates, pigments, low molecular weight proteins and anti-nutritional factors. The molecular weight cut-off of the membrane is usually chosen to ensure retention of a significant proportion of the protein in the solution, while permitting contaminants to pass through having regard to the different membrane materials and configurations.

The concentrated hemp protein solution then may be subjected to a diafiltration step using water or a dilute saline solution. The diafiltration solution may be at its natural pH or at a pH equal to that of the protein solution being diafiltered or at any pH value in between. Such diafiltration may be effected using from about 1 to about 40 volumes of diafiltration solution, preferably about 2 to about 25 volumes of diafiltration solution. In the diafiltration operation, further quantities of contaminants are removed from the aqueous hemp protein solution by passage through the membrane with the permeate. This purifies the aqueous protein solution and may also reduce its viscosity. The diafiltration operation may be effected until no significant further quantities of contaminants or visible colour are present in the permeate or until the retentate has been sufficiently purified so as, when dried, to provide a hemp protein isolate with a protein content of at least about 90 wt % (N×6.25) d.b. Such diafiltration may be effected using the same membrane as for the concentration step. However, if desired, the diafiltration step may be effected using a separate membrane with a different molecular weight cut-off, such as a membrane having a molecular weight cut-off in the range of about 1,000 to about 1,000,000 Daltons, preferably about 1,000 to about 100,000 Daltons, having regard to different membrane materials and configuration.

Alternatively, the diafiltration step may be applied to the acidified aqueous protein solution prior to concentration or to the partially concentrated acidified aqueous protein solution. Diafiltration may also be applied at multiple points during the concentration process. When diafiltration is applied prior to concentration or to the partially concentrated solution, the resulting diafiltered solution may then be additionally concentrated. The viscosity reduction achieved by diafiltering multiple times as the protein solution is concentrated may allow a higher final, fully concentrated protein concentration to be achieved. This reduces the volume of material to be dried.

The concentration step and the diafiltration step may be effected herein in such a manner that the hemp protein product subsequently recovered contains less than about 90 wt % protein (N×6.25) d.b., such as at least about 60 wt % protein (N×6.25) d.b. By partially concentrating and/or partially diafiltering the aqueous hemp protein solution, it is possible to only partially remove contaminants. This protein solution may then be dried to provide a hemp protein product with lower levels of purity. The hemp protein product is still highly soluble and able to produce protein solutions, preferably clear protein solutions under acidic conditions.

An antioxidant may be present in the diafiltration medium during at least part of the diafiltration step. The antioxidant may be any convenient antioxidant, such as sodium sulfite or ascorbic acid. The quantity of antioxidant employed in the diafiltration medium depends on the materials employed and may vary from about 0.01 to about 1 wt %, preferably about 0.05 wt %. The antioxidant serves to inhibit the oxidation of any phenolics present in the hemp protein solution.

The optional concentration step and the optional diafiltration step may be effected at any convenient temperature, generally about 2° to about 65, preferably about 20° to about 35° C., and for the period of time to effect the desired degree of concentration and diafiltration. The temperature and other conditions used to some degree depend upon the membrane equipment used to effect the membrane processing, the desired protein concentration of the solution and the efficiency of the removal of contaminants to the permeate.

The optionally concentrated and optionally diafiltered protein solution may be subject to a further defatting operation, if required, as described in U.S. Pat. Nos. 5,844,086 and 6,005,076. Alternatively, defatting of the optionally concentrated and optionally diafiltered protein solution may be achieved by any other convenient procedure.

The optionally concentrated and optionally diafiltered aqueous protein solution may be treated with an adsorbent, such as powdered activated carbon or granulated activated carbon, to remove colour and/or odour compounds. Such adsorbent treatment may be carried out under any convenient conditions, generally at the ambient temperature of the protein solution. For powdered activated carbon, an amount of about 0.025% to about 5% w/v, preferably about 0.05% to about 2% w/v, is employed. The adsorbent may be removed from the hemp protein solution by any convenient means, such as by filtration.

The optionally concentrated and optionally diafiltered aqueous hemp protein solution may be dried by any convenient technique, such as spray drying or freeze drying. A pasteurization step may be effected on the hemp protein solution prior to drying. Such pasteurization may be effected under any desired pasteurization conditions. Generally, the optionally concentrated and optionally diafiltered hemp protein solution is heated to a temperature of about 55° to about 70° C., preferably about 60° to about 65° C., for about 30 seconds to about 60 minutes, preferably about 10 minutes to about 15 minutes. The pasteurized hemp protein solution then may be cooled for drying, preferably to a temperature of about 25° to about 40° C.

The dry hemp protein product has a protein content in excess of about 60 wt % (N×6.25) d.b. Preferably, the dry hemp protein product is an isolate with a high protein content, in excess of about 90 wt % protein, preferably at least about 100 wt % (N×6.25) d.b.

The hemp protein product produced herein is soluble in an acidic aqueous environment, making the product well suited for incorporation into beverages, particularly powdered beverages, but also ready-to-drink carbonated and uncarbonated beverages, to provide protein fortification thereto. Such beverages have a wide range of acidic pH values, ranging from about 2.5 to about 5. The hemp protein product provided herein may be added to such beverages in any convenient quantity to provide protein fortification to such beverages, for example, at least about 5 g of the hemp protein per serving. For powdered beverages, the hemp protein product may be blended with dried beverage prior to reconstitution of the beverage by dissolution in water. In some cases, modification to the normal formulation of the beverages to tolerate the composition of the invention may be necessary where components present in the beverage may adversely affect the ability of the composition of the invention to remain dissolved in the beverage.

### EXAMPLES

### Example 1

This Example illustrates the production of the hemp protein isolate.

22.5 kg of ground hemp press cake was combined with 150 L of 0.15 M CaCl₂ solution at 25.8° C. and agitated for 30 minutes to provide an aqueous protein solution. The residual ground hemp press cake was removed and the resulting protein solution was clarified by centrifugation and filtration to produce a filtrate having a protein content of 1.31% by weight.

The filtrate was then diluted with reverse osmosis purified water and the pH of the sample lowered to 2.68 with HCl that had been diluted with an equal volume of water. The diluted and acidified protein solution had a protein content of 0.88 wt %.

The diluted and acidified protein solution was reduced in volume from 160 L to 7 L by concentration on a polyethersulfone (PES) membrane, having a molecular weight cut-off of 100,000 Daltons, operated at a temperature of approximately 30° C. The concentrated, acidified protein solution, with a protein content of 10.51 wt %, was diafiltered with 35 L of reverse osmosis purified water, with the diafiltration operation conducted at approximately 30° C. The resulting 7.38 kg of diafiltered protein solution had a protein content of 9.65 wt % and represented a yield of 50.4 wt % of the diluted and acidified protein solution that was further processed. The protein solution was then dried to yield a product found to have a protein content of 108.31 wt % (N×6.25) d.b. The product was given designation H001-H24-11A H701.

### Example 2

This Example contains an evaluation of the phytic acid content of the hemp protein isolate produced by the method of Example 1 as well as the commercial hemp protein concentrate Hemp Pro 70 (Manitoba Harvest, Winnipeg, MB), the protein content of which was determined by combustion analysis using a Leco Nitrogen Determinator to be 65.76% d.b.

Phytic acid content was determined using the method of Latta and Eskin (J. Agric. Food Chem., 28: 1313-1315).

The phytic acid content of the H001-H24-11A H701 was 0.22% d.b. and that of Hemp Pro 70 was 1.43% d.b.

### Example 3

This Example illustrates the colour of the hemp protein isolate prepared by the method of Example 1 and the commercial hemp protein concentrate Hemp Pro 70 in solution and in dry powder form.

Solutions of H001-H24-11A H701 and Hemp Pro 70 were prepared by dissolving sufficient protein powder to supply 0.48 g of protein in 15 ml of RO water. The pH of the solution was measured with a pH meter and the colour and clarity assessed using a HunterLab ColorQuest XE instrument operated in transmission mode. The results are shown in the following Table 1:

TABLE 1
pH and HunterLab readings for solutions of
H001-H24-11A H701 and Hemp Pro 70

| Sample | pH | L* | a* | b* | Haze |
|---|---|---|---|---|---|
| H001-H24-11A H701 | 3.24 | 97.69 | −0.21 | 10.54 | 67.5 |
| Hemp Pro 70 | 7.45 | 1.53 | 3.64 | 2.42 | 96.5 |

As may be seen from the results in Table 1, the solution of H001-H24-11 A H701 was light in colour and translucent. The solution of Hemp Pro 70 was darker, more red, less yellow and had a higher haze level than the solution of H001-H24-11A H701.

The colour of the dry powders was assessed using the HunterLab ColorQuest XE instrument operated in reflectance mode. The colour values are set forth in the following Table 2:

TABLE 2
HunterLab scores for H001-H24-11A H701 and
Hemp Pro 70 dry powders

| Sample | L* | a* | b* |
|---|---|---|---|
| H001-H24-11A H701 | 85.45 | 0.90 | 9.58 |
| Hemp Pro 70 | 52.29 | 2.78 | 24.44 |

As may be seen from the results presented in Table 2, the H001-H24-11A H701 powder was lighter, less red and less yellow than the Hemp Pro 70 powder.

### Example 4

This Example contains an evaluation of the solubility in water of the hemp protein isolate produced by the method of Example 1 as well as the commercial hemp protein concentrate Hemp Pro 70, a product promoted as being water soluble. Solubility was tested based on protein solubility (termed protein method, a modified version of the procedure of Morr et al., J. Food Sci. 50:1715-1718) and total product solubility (termed pellet method).

Sufficient protein powder to supply 0.5 g of protein was weighed into a beaker and then a small amount of reverse osmosis (RO) purified water was added and the mixture stirred until a smooth paste formed. Additional water was then added to bring the volume to approximately 45 ml. The contents of the beaker were then slowly stirred for 60 minutes using a magnetic stirrer. The pH was determined immediately after dispersing the protein and was adjusted to the appropriate level (2, 3, 4, 5, 6 or 7) with diluted NaOH or HCl. A sample was also prepared at natural pH. For the pH adjusted samples, the pH was measured and corrected periodically during the 60 minutes stirring. After the 60 minutes of stirring, the samples were made up to 50 ml total volume with RO water, yielding a 1% w/v protein dispersion. The protein content of the dispersions was measured by combustion analysis using a Leco Nitrogen Determinator. Aliquots (20 ml) of the dispersions were then transferred to pre-weighed centrifuge tubes that had been dried overnight in a 100° C. oven then cooled in a desiccator and the tubes capped. The samples were centrifuged at 7,800 g for 10 minutes, which sedimented insoluble material and yielded a supernatant. The protein content of the supernatant was measured by combustion analysis and then the supernatant and the tube lids were discarded and the pellet material dried overnight in an oven set at 100° C. The next morning the tubes were transferred to a desiccator and allowed to cool. The weight of dry pellet material was recorded. The dry weight of the initial protein powder was calculated by multiplying the weight of powder used by a factor of ((100−moisture content of the powder (%))/100). Solubility of the product was then calculated two different ways:

Solubility(protein method)(%)=(% protein in supernatant/% protein in initial dispersion)×100 1)

Solubility(pellet method)(%)=(1−(weight dry insoluble pellet material/((weight of 20 ml of dispersion/weight of 50 ml of dispersion)×initial weight dry protein powder)))×100 2)

Values calculated to be greater than 100% were expressed as 100%.

The solubility results are set forth in the following Table 3. The natural pH for the sample of H001-H24-11A H701 was 3.31. The natural pH for the sample of Hemp Pro 70 was 7.69.

TABLE 3
Solubility of H001-H24-11A H701 and Hemp Pro 70 at different
pH values as determined by protein method

| | Solubility (%) | | | | | | |
|---|---|---|---|---|---|---|---|
| product | pH 2 | pH 3 | pH 4 | pH 5 | pH 6 | pH 7 | Nat. pH |
| H001-H24-11A | 94.4 | 100 | 99.1 | 81.7 | 67.0 | 63.7 | 99.1 |
| H701 | | | | | | | |
| Hemp Pro 70 | 53.2 | 47.7 | 12.7 | 10.3 | 10.4 | 15.1 | 16.2 |

TABLE 4
Solubility of H001-H24-11A H701 and Hemp Pro 70 at different
pH values as determined by pellet method

| | Solubility (%) | | | | | | |
|---|---|---|---|---|---|---|---|
| product | pH 2 | pH 3 | pH 4 | pH 5 | pH 6 | pH 7 | Nat. pH |
| H001-H24-11A | 99.7 | 99.2 | 98.5 | 77.6 | 66.5 | 59.3 | 98.2 |
| H701 | | | | | | | |
| Hemp Pro 70 | 50.0 | 46.0 | 27.2 | 24.3 | 25.6 | 26.9 | 30.3 |

As may be seen from the results presented in Tables 3 and 4, the H001-H24-11A H701 was highly soluble in the pH range 2 to 4. The Hemp Pro 70 was only partially soluble at all pH values tested.

### Example 5

This Example contains an evaluation of the clarity in water of the hemp protein isolate produced by the method of Example 1 as well as the commercial hemp protein concentrate Hemp Pro 70.

The clarity of the 1% w/v protein dispersions prepared as described in Example 4 was assessed by measuring the absorbance at 600 nm (water blank), with a lower absorbance score indicating greater clarity. Analysis of the samples on the HunterLab ColorQuest XE instrument in transmission mode also provided a percentage haze reading, another measure of clarity.

The clarity results are set forth in the following Tables 5 and 6.

TABLE 5
Clarity of H001-H24-11A H701 and Hemp Pro 70 solutions
at different pH values as assessed by A600

| | A600 | | | | | | |
|---|---|---|---|---|---|---|---|
| Product | pH 2 | pH 3 | pH 4 | pH 5 | pH 6 | pH 7 | Nat. pH |
| H001-H24-11A | 0.011 | 0.043 | 0.110 | 1.542 | 2.477 | 2.567 | 0.058 |
| H701 | | | | | | | |
| Hemp Pro 70 | 2.644 | 2.744 | 3.153 | 3.063 | 3.031 | 3.023 | 3.130 |

TABLE 6
Clarity of H001-H24-11A H701 and Hemp Pro 70 solutions at
different pH values as assessed by HunterLab analysis

| | HunterLab haze reading (%) | | | | | | |
|---|---|---|---|---|---|---|---|
| Product | pH 2 | pH 3 | pH 4 | pH 5 | pH 6 | pH 7 | Nat. pH |
| H001-H24-11A | 0.0 | 11.2 | 30.0 | 94.8 | 95.5 | 95.5 | 16.5 |
| H701 | | | | | | | |
| Hemp Pro 70 | 96.2 | 96.0 | 97.2 | 99.0 | 97.7 | 96.7 | 96.3 |

As may be seen from the results in Tables 5 and 6, the greatest solution clarity for the H001-H24-11A H701 was observed at the lower pH values. The Hemp Pro 70 provided very cloudy solutions at all the pH values tested.

### Example 6

This Example contains an evaluation of the protein solubility in a soft drink and sports drink of the hemp protein isolate produced by the method of Example 1 and the commercial hemp protein concentrate Hemp Pro 70. The solubility was determined with the protein added to the beverages with no pH correction and again with the pH of the protein fortified beverages adjusted to the level of the original beverages.

When the solubility was assessed with no pH correction, a sufficient amount of protein powder to supply 1 g of protein was weighed into a beaker and a small amount of beverage was added and stirred until a smooth paste formed. Additional beverage was added to bring the volume to 50 ml, and then the solutions were stirred slowly on a magnetic stirrer for 60 minutes to yield a 2% protein w/v dispersion. The protein content of the samples was determined by combustion analysis using a LECO Nitrogen Determinator then an aliquot of the protein containing beverages was centrifuged at 7,800 g for 10 minutes and the protein content of the supernatant measured.

Solubility (%)=(% protein in supernatant/% protein in initial dispersion)×100

Values calculated to be greater than 100% were expressed as 100%.

When the solubility was assessed with pH correction, the pH of the soft drink (Sprite) (3.59) and sports drink (Orange Gatorade) (3.29) without protein was measured. A sufficient amount of protein powder to supply 1 g of protein was weighed into a beaker and a small amount of beverage was added and stirred until a smooth paste formed. Additional beverage was added to bring the volume to approximately 45 ml, and then the solutions were stirred slowly on a magnetic stirrer for 60 minutes. The pH of the protein containing beverages was determined immediately after dispersing the protein and was adjusted to the original no-protein pH with HCl or NaOH solution as necessary. The pH was measured and corrected periodically during the 60 minutes stirring. After the 60 minutes of stirring, the total volume of each solution was brought to 50 ml with additional beverage, yielding a 2% protein w/v dispersion. The protein content of the samples was determined by combustion analysis using a Leco Nitrogen Determinator then an aliquot of the protein containing beverages was centrifuged at 7,800 g for 10 minutes and the protein content of the supernatant measured.

Solubility (%)=(% protein in supernatant/% protein in initial dispersion)×100

Values calculated to be greater than 100% were expressed as 100%.

The results obtained are set forth in the following Table 7.

TABLE 7
Solubility of H001-H24-11A H701 in Sprite and Orange Gatorade

| | No pH correction | | pH correction | |
|---|---|---|---|---|
| | | Solubility in | | Solubility in |
| | | Orange | | Orange |
| | Solubility in | Gatorade | Solubility in | Gatorade |
| Product | Sprite (%) | (%) | Sprite (%) | (%) |
| H001-H24-11A | 96.6 | 100 | 100 | 98.1 |
| H701 | | | | |
| Hemp Pro 70 | 8.7 | 9.0 | 32.3 | 39.5 |

The natural pH of the H701 was similar to that of the beverages so protein addition had only a minor effect on beverage pH. As can be seen from the results of Table 7 the H001-H24-11A H701 was highly soluble in both the Sprite and the Orange Gatorade, with and without pH correction and was much more soluble than the Hemp Pro 70 protein.

### Example 7

This Example contains an evaluation of the clarity in a soft drink and sports drink of the hemp protein isolate produced by the method of Example 1.

The clarity of the 2% w/v protein dispersions prepared in soft drink (Sprite) and sports drink (Orange Gatorade) in Example 6 were assessed using the spectrophotometric and HunterLab methods as described in Example 5. In this case however, the spectrophotometer was blanked with the appropriate beverage.

The results obtained are set forth in the following Tables 8 and 9.

TABLE 8
A600 readings for H001-H24-11A H701 in Sprite
and Orange Gatorade

| | No pH correction | | pH correction | |
|---|---|---|---|---|
| | | A600 in | | A600 in |
| | | Orange | | Orange |
| | A600 in | Gatorade | A600 in | Gatorade |
| Product | Sprite (%) | (%) | Sprite (%) | (%) |
| H001-H24-11A H701 | 0.189 | 0.352 | 0.530 | 0.444 |
| Hemp Pro 70 | nd | 3.596 | nd | 3.157 |

nd = not determined

TABLE 9
HunterLab haze readings for H001-H24-11A H701
in Sprite and Orange Gatorade

| | No pH correction | | pH correction | |
|---|---|---|---|---|
| | | Haze in | | Haze in |
| | Haze in | Orange | Haze in | Orange |
| Product | Sprite (%) | Gatorade (%) | Sprite (%) | Gatorade (%) |
| no protein | 0.0 | 50.9 | | |
| H001-H24-11A | 58.2 | 88.6 | 88.7 | 91.1 |
| H701 | | | | |
| Hemp Pro 70 | 98.4 | 99.3 | 96.4 | 95.9 |

As can be seen from the results in Tables 8 and 9, despite the excellent protein solubility, the H001-H24-11A H701 contributed haze to the Sprite and Orange Gatorade. However, samples prepared with Hemp Pro 70 were cloudier than the samples prepared with H001-H24-11A H701.

### Example 8

This Example illustrates a comparison of the flavor of the H701, prepared as described in Example 1, with that of the commercial hemp protein concentrate Hemp Pro 70, with the evaluation done at low pH.

Samples were prepared for sensory evaluation by dissolving sufficient protein powder to supply 5 g of protein in 250 ml purified drinking water. The pH of the solution of H701 was determined to be 3.31. Food grade HCl was added to the solution of Hemp Pro 70 to lower the pH from 7.73 to 3.31. An informal panel of seven panelists was asked to blindly compare the samples and indicate which sample was more bland in flavour, and of which sample they preferred the flavour.

Six out of seven panelists found the flavour of the H701 to be more bland, while seven out of seven panelists preferred the flavour of the H701.

### Example 9

This Example illustrates a comparison of the flavor of the H701, prepared as described in Example 1, with that of the commercial hemp protein concentrate Hemp Pro 70, with the evaluation done at near neutral pH.

Samples were prepared for sensory evaluation by dissolving sufficient protein powder to supply 5 g of protein in 250 ml purified drinking water. The pH of the solution of Hemp Pro 70 was determined to be 7.72. Food grade NaOH was added to the solution of H701 to raise the pH from 3.23 to 7.72. An informal panel of seven panelists was asked to blindly compare the samples and indicate which sample was more bland in flavour, and of which sample they preferred the flavour.

Four out of seven panelists found the flavour of the H701 to be more bland, while four out of seven panelists preferred the flavour of the H701.

### SUMMARY OF THE DISCLOSURE

In summary of this disclosure, the present invention provides a novel hemp protein product, which may be in the form of an isolate, which is completely soluble at acid pH and is useful in the protein fortification of aqueous systems, including soft drinks and sports drinks, particularly powdered versions of these drinks, without leading to protein precipitation. Modifications are possible within the scope of this invention.

What we claim is:

1. A method of producing a hemp protein product having a protein content of at least about 60 wt %, preferably at least about 90 wt %, (N×6.25) on a dry weight basis, which comprises:
(a) extracting a hemp protein source with an aqueous calcium salt solution to cause solubilization of hemp protein from the protein source and to form an aqueous hemp protein solution,
(b) at least partially separating the aqueous hemp protein solution from residual hemp protein source,
(c) optionally diluting the aqueous hemp protein solution,
(d) adjusting the pH of the aqueous hemp protein solution to a pH of about 1.5 to about 4.4 to produce an acidified aqueous hemp protein solution,
(e) optionally clarifying the acidified hemp protein solution if it is not already clear,
(f) alternatively from steps (b) to (e), optionally diluting and then adjusting the pH of the combined aqueous hemp protein solution and residual hemp protein source to a pH of about 1.5 to about 4.4 then separating the acidified aqueous hemp protein solution from residual hemp protein source,
(g) optionally concentrating the aqueous hemp protein solution while maintaining the ionic strength substantially constant by a selective membrane technique,
(h) optionally diafiltering the optionally concentrated hemp protein solution, and
(i) optionally drying the optionally concentrated and optionally diafiltered hemp protein solution.

2. The method of claim 1 wherein said aqueous calcium salt solution is an aqueous calcium chloride solution.

3. The method of claim 2 wherein said aqueous calcium chloride solution has a concentration less than about 1.0 M.

4. The method of claim 3 wherein said concentration is about 0.10 to about 0.15 M.

5. The method of claim 1 wherein said extraction step (a) is effected at a temperature of about 1° to about 65° C., preferably about 15° to about 65° C., more preferably 20° to about 35° C.

6. The method of claim 1 wherein said extraction with aqueous calcium salt solution is conducted at a pH of about 4.5 to about 11.

7. The method of claim 6 wherein said pH is about 5 to about 7.

8. The method of claim 1 wherein said aqueous hemp protein solution has a protein concentration of about 5 to about 50 g/L.

9. The method of claim 8 wherein said protein concentration is about 10 to about 50 g/L.

10. The method of claim 1 wherein said aqueous calcium salt solution contains an antioxidant.

11. The method of claim 1 wherein, following said separation step (b) and prior to said optional dilution step (c) or in step (f) after said separation step, said aqueous hemp protein solution is treated with an adsorbent to remove colour and/or odour compounds from the aqueous hemp protein solution.

12. The method of claim 1 wherein said aqueous hemp protein solution is diluted in step (c) or (f) with about 0.1 to about 10 volumes of aqueous diluent to a conductivity of less than about 105 mS.

13. The method of claim 12 wherein said aqueous hemp protein solution is diluted in step (c) or (f) with about 0.5 to about 2 volumes of aqueous diluent to provide a conductivity of said hemp protein solution of about 4 to about 21 mS.

14. The method of claim 12 wherein said aqueous diluent has a temperature of about 1° to about 100° C.

15. The method of claim 14 wherein said temperature is about 15° to about 65° C.

16. The method of claim 15 wherein said temperature is about 20° to about 35° C.

17. The method of claim 1 wherein said acidified hemp protein solution has a conductivity of less than about 110 mS if diluted or less than 115 mS if not diluted.

18. The method of claim 17 wherein said conductivity is about 4 to about 26 mS.

19. The method of claim 1 wherein the pH of said aqueous hemp protein solution is adjusted in step (d) or (f) to about pH 2 to about 4.

20. The method of claim 1 wherein the acidified hemp protein solution is subjected to step (e).

21. The method of claim 1 wherein said acidified aqueous hemp protein solution is dried to provide a hemp protein product having a protein content of at least about 60 wt % (N×6.25) d.b.

22. The method of claim 1 wherein said acidified aqueous hemp protein solution is subjected to step (g) to produce a concentrated acidified hemp protein solution having a protein concentration of about 50 to about 300 g/L.

23. The method of claim 22 wherein said concentrated acidified hemp protein solution has a protein concentration of about 100 to about 200 g/L.

24. The method of claim 22 wherein said concentration step (g) is effected by ultrafiltration using a membrane having a molecular weight cut-off of about 1,000 to about 1,000,000 Daltons.

25. The method of claim 24 wherein said membrane has a molecular weight cut-off of about 1,000 to about 100,000 Daltons.

26. The method of claim 1 wherein said diafiltration step (h) is effected using water, acidified water, dilute saline or acidified dilute saline on the acidified hemp protein solution before or after partial or complete concentration thereof.

27. The method of claim 26 wherein said diafiltration step (h) is effected using about 1 to about 40 volumes of diafiltration solution.

28. The method of claim 27 wherein said diafiltration step (h) is effected using about 2 to about 25 volumes of diafiltration solution.

29. The method of claim 26 wherein said diafiltration step (h) is effected until no significant further quantities of contaminants or visible colour are present in the permeate.

30. The method of claim 26 wherein said diafiltration step (h) is effected until the retentate has been sufficiently purified so as, when dried, to provide a hemp protein isolate with a protein content of at least about 90 wt % (N×6.25) d.b.

31. The method of claim 26 wherein said diafiltration step (h) is effected using a membrane having a molecular weight cut-off of about 1,000 to about 1,000,000 Daltons.

32. The method of claim 31 wherein said membrane has a molecular weight cut-off of about 1,000 to about 100,000 Daltons.

33. The method of claim 26 wherein an antioxidant is present in the diafiltration medium during at least part of the diafiltration step (h).

34. The method of claim 22 or claim 26 wherein said concentration step (g) and optional diafiltration step (h) are carried out at a temperature of about 2° to about 65° C.

35. The method of claim 34 wherein said temperature is about 20° to about 35° C.

36. The method of claim 1 wherein said acidified aqueous hemp protein solution is subjected to steps (g) and (h) to produce a concentrated and/or diafiltered acidified hemp protein solution which, when dried, provides a hemp protein product having a protein concentration of at least about 60 wt % (N×6.25) d.b.

37. The method of claim 22 or claim 26 wherein said optionally concentrated and optionally diafiltered acidified hemp protein solution is treated with an adsorbent to remove colour and/or odour compounds.

38. The method of claim 22 or claim 26 wherein said optionally concentrated and optionally diafiltered acidified hemp protein solution is pasteurized prior to drying.

39. The method of claim 38 wherein said pasteurization step is effected at a temperature of about 55° to about 70° C. for about 30 seconds to about 60 minutes.

40. The method of claim 39 wherein said pasteurization step is effected at a temperature of about 60° to about 65° C. for about 10 to about 15 minutes.

41. The method of claim 30 wherein said optionally concentrated and diafiltered acidified hemp protein solution is subjected to step (i) to provide a hemp protein isolate having a protein content of at least about 90 wt % (N×6.25) d.b.

42. The method of claim 41 wherein said hemp protein isolate has a protein content of at least about 100 wt % (N×6.25) d.b.

43. A hemp protein product having a protein content of at least about 60 wt % (N×6.25) d.b. which is water soluble at acid pH values of less than about 4.4.

44. The hemp protein product of claim 43 having a protein content of at least about 90 wt % (N×6.25) d.b.

45. The protein product of claim 43 having a protein content of at least about 100 wt % (N×6.25) d.b.

46. The hemp protein product of claim 43 which has a bland flavour.

47. The hemp protein product of claim 43 which is blended with water-soluble powdered materials for the production of aqueous solutions of the blend.

48. The blend of claim 47 which is a powdered beverage.

49. An aqueous solution of the hemp protein product of claim 43 having a pH of less than about 4.4.

50. The aqueous solution of claim 49 which is a beverage.

51. An aqueous solution of the hemp protein product of claim 43 having a pH of about 6 to about 8.

52. The aqueous solution of claim 51 which is a beverage.

53. The aqueous solution of claim 51 which is used in the production of a dairy analogue or dairy alternative product or a product that is a blend of plant and dairy ingredients.
